(19) United States
(12) Patent Application Publication
(10) Pub. No.: US 2025/0177463 A1
(43) Pub. Date: Jun. 5, 2025

(54) Equine Supplement Composition

(71) Applicant: Aniko Nelson, Bozeman, MT (US)

(72) Inventor: Aniko Nelson, Bozeman, MT (US)

(21) Appl. No.: 18/964,432

(22) Filed: Nov. 30, 2024

Related U.S. Application Data
(60) Provisional application No. 63/604,872, filed on Nov. 30, 2023.

(51) Int. Cl. A61K 36/185; A61K 31/10; A61K 31/198; A61K 35/748; A61K 36/28; A61K 36/48; A61K 36/484; A61K 36/81; A61K 36/815

(52) U.S. Cl. CPC A61K 36/185; A61K 31/10; A61K 31/198; A61K 35/748; A61K 36/28; A61K 36/48; A61K 36/484; A61K 36/577; A61K 36/81; A61K 36/815

(57) ABSTRACT

Disclosed are equine supplement compositions for enhancing the health and well-being of horses. For instance, a composition may include a first portion containing one or more components selected from slippery elm, milk thistle, spirulina, marshmallow root, licorice root, alfalfa leaf, and ashwaganda; and a second portion containing one or more components selected from lysine, MSM, and goji berry. Additionally, a second composition may include an oil component selected from flax seed oil and coconut oil, combined with a honey component comprising manuka honey. Furthermore, a third composition may include a vitamin E component and a selenium component. The supplemental compositions provides a novel, cost-effective, simple, effective, efficient, and sustainable solution for supporting the overall health, performance, and longevity of horses by providing essential nutrients and beneficial compounds that target various aspects of equine health.

### CROSS-REFERENCE TO RELATED APPLICATION(S)

This application claims the benefit of U.S. Provisional Application No. 63/604,872, filed Nov. 30, 2023.

The contents of each of the above referenced applications are hereby incorporated by reference in their entirety.

### TECHNICAL FIELD

Various aspects of the present disclosure relate generally to equine supplement compositions and, more particularly, to equine supplement compositions for daily supplements, pre-race pastes, top dressings, and/or EDM compositions.

### BACKGROUND

The present disclosure relates generally to the field of equine health and nutrition, and more particularly to equine supplemental compositions for addressing various health concerns and promoting overall well-being in horses.

In the realm of equine health, a variety of issues can arise, such as digestive problems, immune system deficiencies, joint discomfort, and oxidative stress, among others. To maintain optimal health and performance, horses often require supplementation with various nutrients, vitamins, and minerals. However, conventional supplements may not be specifically tailored to address the unique needs of equines and may lack the ideal combination of ingredients to effectively target and alleviate these health concerns.

Several challenges exist in the current state of equine supplementation. One such challenge is to create a balanced and comprehensive supplement that addresses multiple health issues simultaneously. Additionally, it is important to ensure that the ingredients used in such supplements are safe, natural, and easily digestible for horses. Moreover, there is a need for supplements that can be easily administered and incorporated into a horse's daily diet without causing adverse effects or requiring significant changes to their feeding regimen.

Traditional methods of addressing equine health issues often involve the use of pharmaceutical drugs, which may have undesirable side effects and may not always be the best option for long-term health and well-being. Furthermore, these methods may not always provide a comprehensive solution that addresses multiple health concerns simultaneously. As such, there is a need for alternative, natural solutions that can effectively address the various health challenges faced by horses.

Various equine supplements have been developed that target specific health issues, such as digestive support, joint health, or immune system support. However, these supplements may not comprehensively address the full range of health concerns encountered by horses. Additionally, the supplements may not contain the ideal combination of ingredients to effectively target and alleviate these health concerns in a safe and natural manner.

In view of the foregoing, there is a need for an equine supplemental composition that addresses multiple health concerns in horses, including digestive problems, immune system deficiencies, joint discomfort, and oxidative stress. The present disclosure aims to provide a comprehensive solution that utilizes a combination of natural ingredients specifically tailored to address these issues in a safe, effective, and easily digestible manner. Furthermore, the disclosure aims to offer a versatile supplement that can be easily administered and incorporated into a horse's daily diet to promote overall health and well-being.

The present disclosure is directed to overcoming one or more of these above-referenced challenges.

### SUMMARY OF THE DISCLOSURE

According to certain aspects of the disclosure, systems, methods, and computer readable memory are disclosed for equine supplement compositions.

In some cases, an equine supplemental composition includes: a first portion including one or combinations of: a slippery elm component, a milk thistle component, a spirulina component, a marshmallow root component, a licorice root component, an alfalfa leaf component, an ashwaganda component; and a second portion including one or combinations of: a lysine component, a MSM component, and a goji berry component.

In some cases, an equine supplemental composition includes: equal parts of each of: a milk thistle component, a licorice root component, and a slippery elm component.

In some cases, an equine supplemental composition includes: an oil component and a honey component.

In some cases, an equine supplemental composition includes: a vitamin E component, a selenium component, and one or combinations of: a slippery elm component, a milk thistle component, a spirulina component, a marshmallow root component, a licorice root component, an alfalfa leaf component, an ashwaganda component, a lysine component, a MSM component, and a goji berry component.

Additional objects and advantages of the disclosed technology will be set forth in part in the description that follows, and in part will be apparent from the description, or may be learned by practice of the disclosed technology.

It is to be understood that both the foregoing general description and the following detailed description are exemplary and explanatory only and are not restrictive of the disclosed technology, as claimed.

### DETAILED DESCRIPTION

Various aspects of the present disclosure relate generally to equine supplement compositions.

In some cases, an equine supplemental composition may include various components to support and enhance the overall health and wellbeing of horses.

In some cases, an equine supplemental composition may include a first portion comprising one or more of a slippery elm component, a milk thistle component, a spirulina component, a marshmallow root component, a licorice root component, an alfalfa leaf component, and an ashwaganda component, and a second portion comprising one or more of a lysine component, a MSM component, and a goji berry component.

In some cases, an equine supplemental composition may include an oil component selected from the group consisting of flax seed oil and coconut oil, and a honey component comprising manuka honey.

In some cases, an equine supplemental composition is provided, wherein the first portion comprises one or more of the aforementioned components, and the second portion comprises the lysine component, the MSM component, and the goji berry component.

In some cases, an equine supplemental composition is provided, wherein the oil component comprises a combination of flax seed oil and coconut oil.

In some cases, an equine supplemental composition is provided, comprising a vitamin E component and a selenium component.

In some cases, the equine supplemental composition may also include one or more of the following components: a slippery elm component, a milk thistle component, a spirulina component, a marshmallow root component, a licorice root component, an alfalfa leaf component, an ashwaganda component, a lysine component, a MSM component, and a goji berry component.

The equine supplemental compositions of the present disclosure may provide various advantages, such as supporting the overall health and wellbeing of horses, addressing specific nutritional needs, and offering a comprehensive and customizable approach to equine supplementation.

The present disclosure relates to an equine supplemental composition for promoting the health and well-being of horses. The composition comprises a first portion containing one or more of a slippery elm component, a milk thistle component, a spirulina component, a marshmallow root component, a licorice root component, an alfalfa leaf component, and an ashwaganda component. These components may be sourced from natural or synthetic sources and may be present in various forms such as powders, extracts, or whole plant materials.

The first portion of the equine supplemental composition may provide various health benefits to horses. For example, the slippery elm component may help soothe the digestive system. The milk thistle component may support liver function. The spirulina component may provide antioxidants and essential nutrients. The marshmallow root component may aid in digestion. The licorice root component may support respiratory and immune system health. The alfalfa leaf component may provide vitamins and minerals. The ashwaganda component may help reduce stress and promote overall well-being.

The second portion of the equine supplemental composition may include one or more of a lysine component, a MSM component, and a goji berry component. The lysine component may be beneficial in promoting muscle growth and repair. The MSM component may support joint health and reduce inflammation. The goji berry component may provide additional antioxidants and nutrients to support overall health.

In some cases, the second portion of the equine supplemental composition may include all three components: the lysine component, the MSM component, and the goji berry component. This combination may provide a synergistic effect in promoting overall health and well-being in horses.

In some cases, an equine supplemental composition may include an oil component selected from the group consisting of flax seed oil and coconut oil, and a honey component comprising manuka honey. The oil component may be present in the form of a single oil or a combination of both flax seed oil and coconut oil. These oils may provide essential fatty acids, which are important for maintaining healthy skin, coat, and hooves in horses. The honey component, specifically manuka honey, may provide additional benefits to the equine supplemental composition. Manuka honey may provide antibacterial and anti-inflammatory properties, which may help support the overall immune system and promote wound healing in horses.

In some cases, an equine supplemental composition comprising a vitamin E component and a selenium component. Vitamin E and selenium are important antioxidants that help protect cells from damage caused by free radicals. This combination may support overall immune system health and promote healthy muscle function in horses.

In some cases, the equine supplemental composition comprising the vitamin E and selenium components may further include one or more of the components from the first and second portions described previously. These additional components may provide a comprehensive and balanced nutritional supplement for horses, addressing various aspects of equine health and well-being.

The equine supplemental composition may be provided in various forms, such as pellets, powders, liquids, or pastes. The composition may be administered to horses as a daily supplement or as needed to address specific health concerns.

In addition to the embodiments described herein, other variations and modifications may be made to the equine supplemental composition without departing from the scope of the disclosure. For example, additional components may be included to address other aspects of equine health, such as probiotics for digestive support, vitamins and minerals for overall nutrition, or herbs and botanicals for specific health concerns.

The equine supplemental composition may be formulated using various methods known in the art, such as mixing, blending, or extrusion. The composition may also be formulated to provide a specific dosage or concentration of each component, depending on the needs of the individual horse and the desired health benefits.

The equine supplemental compositions of the present disclosure may provide numerous advantages over existing equine supplements. These advantages may include a comprehensive and balanced approach to equine nutrition, addressing various aspects of health and well-being, and the use of natural and beneficial components to support overall health and performance in horses.

Furthermore, the equine supplemental composition may be tailored to address specific health concerns or nutritional needs of individual horses, allowing for a customized approach to equine health management. This may lead to improved overall health, performance, and longevity in horses.

In some cases, a supplemental composition specifically tailored for equines. The composition of the disclosure may include a first portion and a second portion. The first portion contains one or more of a slippery elm component, a milk thistle component, a spirulina component, a marshmallow root component, a licorice root component, an alfalfa leaf component, and an ashwaganda component. These components may be provided individually or in any combination, depending on the specific nutritional needs of the horse.

The use of a slippery elm component in the equine supplemental composition can provide relief from digestive discomfort in horses. Slippery elm, known for its mucilage content, can coat the horse's digestive tract, providing a soothing effect and aiding digestion. The milk thistle component is known for its liver-protective properties. Milk thistle provides a compound called silymarin, which has been shown to promote liver health.

The spirulina component provides a rich source of protein, vitamins, and essential minerals for horses. Marshmallow root is another component that can benefit the equine digestive system by soothing the mucosa and reducing inflammation. Licorice root is known for its benefits in supporting the respiratory system, immune system, and adrenal health of horses. Alfalfa leaf provides a good source of vitamins and minerals necessary for overall equine health. Ashwaganda, an adaptogen, can help the horse's body to adapt to stress, which in turn promotes well-being.

The second portion of the equine supplemental composition comprises one or more of a lysine component, a MSM component, and a goji berry component. Lysine, an essential amino acid, is critical for muscle development and repair in horses. MSM is a potent anti-inflammatory compound that supports joint health. Goji berries are a rich source of antioxidants and essential nutrients, which may enhance overall equine health.

In some cases, a composition made from an oil component selected from flax seed oil and coconut oil, and a honey component comprising manuka honey. Flaxseed oil is a rich source of omega-3 fatty acids, important for maintaining a shiny coat and healthy skin in horses. Coconut oil provides medium-chain triglycerides, which are readily used as an energy source by horses. Manuka honey is known for its anti-inflammatory and antibacterial properties, beneficial to the overall health of horses.

In some cases, an equine supplemental composition made up of a vitamin E component and a selenium component. Vitamin E, a potent antioxidant, protects equine cells from oxidative damage, while selenium supports the immune system and thyroid function in horses.

The equine supplemental composition may further include the components from both the first and second portions. This combination of components provides a comprehensive and balanced nutritional supplement for horses, addressing various aspects of equine health, such as digestive health, joint health, immune function, and overall wellbeing.

In some cases, the equine supplemental composition may include additional components. For example, the composition may include a probiotic component for supporting gut health, a glucosamine component for joint health, or a vitamin C component for immune health. These additional components further enhance the health benefits provided by the equine supplemental composition.

In some cases, the equine supplemental composition may be administered in various forms, such as a pellet, powder, or liquid, depending on the specific needs and preferences of the horse. The composition may be given to horses on a daily basis or as needed to address specific health concerns.

The advantages of the equine supplemental compositions of the present disclosure are numerous. Compared to conventional supplements, the compositions of the present disclosure may provide a comprehensive solution that addresses multiple health concerns simultaneously, using a combination of natural ingredients. This saves the horse owner the time and cost of administering multiple separate supplements.

The equine supplemental compositions may be versatile and can be customized to meet the specific nutritional needs of individual horses. This personalization can result in improved overall health and performance of the horse.

The equine supplemental composition is also easy to administer, as the components are readily digestible and can be incorporated into the horse's regular diet without causing adverse effects or necessitating significant changes to the feeding regimen.

The equine supplemental compositions of the present disclosure may be utilized in various equine health applications. It may be used as a daily supplement to maintain optimal health and performance in horses, or as a targeted treatment for specific health issues, such as digestive problems, joint discomfort, immune deficiencies, and oxidative stress.

Generally, where an ingredient is described as a “component,” the subject ingredient of the component may be provide in various forms, such as powders, extracts, or whole plant materials (as the case may be). Moreover, a “component” may be an “effective amount” (or alternately, a “therapeutically effective amount”) for a targeted dose (e.g., for absorption) alone or combined with other “components.” In some cases, an “effective amount” is an amount that alone, or together with further doses or other components, produces the desired (therapeutic) response. The (therapeutically) effective amount to be used will depend, for example, upon the therapeutic objectives, the route of administration, and the condition of the horse.

The compositions described herein are therefore administered to a horse in an effective amount to produce the desired response. Examples of such responses include (increases or decreases in) thirst, fatigue, headache, dizziness/faintness, loss of appetite, stomach ache, nausea, poor and/or decreased sleep, and elevated heart rate.

### Daily Supplement

Generally, a daily supplement composition may be administrated to a horse for several reasons, including preventive, restorative, or targeted results. In some cases, a daily equine supplemental composition may include: a first portion and a second portion.

The first portion may include one or combinations of: a slippery elm component, a milk thistle component, a spirulina component, a marshmallow root component, a licorice root component, an alfalfa leaf component, an ashwaganda component. The second portion may include one or combinations of: a lysine component, a MSM component, and a goji berry component.

In some cases, the first and second portions may have ratios, by weight to a total weight of the composition, as indicated by Table 1, below.

TABLE 1

| Ratio | Component |
|---|---|
| 0.07 | Slippery Elm |
| 0.02 | Milk Thistle |
| 0.05 | Spirulina |
| 0.12 | Marshmallow Root |
| 0.05 | Licorice Root |
| 0.07 | Alfalfa Leaf |
| 0.07 | Ashwaganda |
| 0.12 | Lysine |
| 0.39 | MSM |
| 0.04 | Goji Berry |
| 1.00 | Total |

Ulmus rubra, Slippery Elm. Slippery Elm may increase mucous secretion, which might be helpful for stomach and intestinal problems. The use of a slippery elm component in the equine supplemental composition can provide relief from digestive discomfort in horses. Slippery elm, known for its mucilage content, can coat the horse's digestive tract, providing a soothing effect and aiding digestion.

Silybum marianum, Milk Thistle. Milk Thistle may include silymarin which is an antioxidant compound taken from milk thistle seeds. Milk Thistle may be a treatment for things including cirrhosis, jaundice, hepatitis, and gallbladder disorders. The milk thistle component is known for its liver-protective properties. Milk thistle provides a compound called silymarin, which has been shown to promote liver health.

Spirulina. Spirulina is a biomass of cyanobacteria (blue-green algae) that can be consumed by horses. The three species may include Arthrospira platensis, A. fusiformis, and A. maxima. The spirulina component provides a rich source of protein, vitamins, and essential minerals for horses.

Althaea officinalis, Marshmallow Root. Marshmallow Root may help form a protective layer on the skin and lining of the digestive tract. Marshmallow Root may also contains chemicals that might decrease cough and fight infections. Marshmallow root is another component that can benefit the equine digestive system by soothing the mucosa and reducing inflammation.

Glycyrrhiza glabra, Glycyrrhiza uralensis, Glycyrrhiza inflata, Licorice Root. Licorice Root may help with digestive problems, cough, and bacterial and viral infections. Licorice Root may also help prevent or reduce sore throat. Licorice root is known for its benefits in supporting the respiratory system, immune system, and adrenal health of horses.

Medicago sativa, Alfalfa Leaf. Alfalfa Leaf is an herb that can be a source of calcium, potassium, phosphorous, iron, and vitamins A, C, E, and K. Alfalfa leaf provides a good source of vitamins and minerals necessary for overall equine health.

Withania somnifera, Ashwaganda. Ashwaganda is an evergreen shrub. Ashwaganda, an adaptogen, can help the horse's body to adapt to stress, which in turn promotes well-being.

Lysine. Lysine is an α-amino acid that is a precursor to many proteins. Lysine, an essential amino acid, is critical for muscle development and repair in horses.

Methylsulfonylmethane, MSM. MSM is a chemical, used to lessen inflammation. MSM is a potent anti-inflammatory compound that supports joint health.

Goji Berry. The sweet fruit of either Lycium barbarum or Lycium chinense. Goji berries are a rich source of antioxidants and essential nutrients, which may enhance overall equine health.

In some cases, the second portion includes the lysine component, the MSM component, and the goji berry component. In some cases, the second portion includes the lysine component and the goji berry component. In some cases, the second portion includes the MSM component and the goji berry component. In some cases, the second portion includes the lysine component and the MSM component.

In some cases, the second portion includes the lysine component with a ratio, by weight to a total weight of the composition, from 0.01 to 0.15.

In some cases, the second portion includes the MSM component with a ratio, by weight to a total weight of the composition, from 0.20 to 0.60.

In some cases, the second portion includes the goji berry component with a ratio, by weight to a total weight of the composition, from 0.001 to 0.10.

In some cases, the first portion includes the slippery elm component, the milk thistle component, the spirulina component, the marshmallow root component, the licorice root component, the alfalfa leaf component, and the ashwaganda component.

In some cases, the first portion includes the slippery elm component and one or combinations of: the milk thistle component, the spirulina component, the marshmallow root component, the licorice root component, the alfalfa leaf component, and the ashwaganda component.

In some cases, the first portion includes the milk thistle component and one or combinations of: the slippery elm component, the spirulina component, the marshmallow root component, the licorice root component, the alfalfa leaf component, and the ashwaganda component.

In some cases, the first portion includes the spirulina component and one or combinations of: the slippery elm component, the milk thistle component, the marshmallow root component, the licorice root component, the alfalfa leaf component, and the ashwaganda component.

In some cases, the first portion includes the marshmallow root component and one or combinations of: the slippery elm component, the milk thistle component, the spirulina component, the licorice root component, the alfalfa leaf component, and the ashwaganda component.

In some cases, the first portion includes the licorice root component and one or combinations of: the slippery elm component, the milk thistle component, the spirulina component, the marshmallow root component, the alfalfa leaf component, and the ashwaganda component.

In some cases, the first portion includes the alfalfa leaf component and one or combinations of: the slippery elm component, the milk thistle component, the spirulina component, the marshmallow root component, the licorice root component, and the ashwaganda component.

In some cases, the first portion includes the ashwaganda component and one or combinations of: the slippery elm component, the milk thistle component, the spirulina component, the marshmallow root component, the licorice root component, and the alfalfa leaf component.

In some cases, the first portion includes the slippery elm component with a ratio, by weight to a total weight of the composition, from 0.01 to 0.20.

In some cases, the first portion includes the milk thistle component with a ratio, by weight to a total weight of the composition, from 0.001 to 0.10.

In some cases, the first portion includes the spirulina component with a ratio, by weight to a total weight of the composition, from 0.01 to 0.10.

In some cases, the first portion includes the marshmallow root component with a ratio, by weight to a total weight of the composition, from 0.01 to 0.20.

In some cases, the first portion includes the licorice root component with a ratio, by weight to a total weight of the composition, from 0.01 to 0.10.

In some cases, the first portion includes the alfalfa leaf component with a ratio, by weight to a total weight of the composition, from 0.01 to 0.20.

In some cases, the first portion includes the ashwaganda component with a ratio, by weight to a total weight of the composition, from 0.01 to 0.20.

### Alternative Daily Supplement

In some cases, an alternative daily equine supplemental composition may include a first portion and a second portion, where the first portion replaces the alfalfa leaf component with a moringa leaf component. This alternative composition may provide additional benefits and may be suitable for horses that have difficulty processing alfalfa.

The first portion of this alternative daily supplement may include one or combinations of: a slippery elm component, a milk thistle component, a spirulina component, a marshmallow root component, a licorice root component, a moringa leaf component, and an ashwaganda component. The second portion may remain the same, including one or combinations of: a lysine component, a MSM component, and a goji berry component.

In some cases, the moringa leaf component may have a ratio, by weight to a total weight of the composition, from 0.01 to 0.20, similar to the ratio previously used for the alfalfa leaf component.

Moringa oleifera, Moringa Leaf. Moringa leaf may offer a wide range of potential benefits for equine health. Studies have shown that moringa leaf may be effective in controlling gastrointestinal parasites in horses, affecting both the larvae and locates of different parasites. The moringa leaf component may contain beneficial bioactive compounds such as tannins, flavonoids, phenolic acids, vitamins, minerals, and saponins.

In some cases, the moringa leaf component may provide a diverse profile of amino acids, which, in combination with the lysine component, may aid in repair and recovery after exercise and work. The moringa leaf component may also offer a balanced mixture of calcium, potassium, and magnesium, which may support healthy electrolyte balance, joints, and bones in horses.

Studies suggest that the moringa leaf component may have potential benefits for milk production, blood iron levels, and healthy hemoglobin in horses. These properties may play a role in supporting cardiovascular health and reducing systemic inflammation.

In some cases, the polyphenols present in the moringa leaf component may help enhance blood flow by potentially reducing blood pressure and increasing nitric oxide production. This may be beneficial in limiting bleeding in the lungs due to bursting capillaries in horses. The increased nitric oxide production may also support immune system function, oxygenation of muscle tissues, blood flow, neural activity, and blood flow to the brain.

The alternative daily supplement composition including the moringa leaf component may offer a more economical option while potentially benefiting a wider range of horses, including those that may have difficulty processing alfalfa. This composition may provide a comprehensive approach to supporting equine health, addressing various aspects such as digestive health, parasite control, muscle repair, electrolyte balance, cardiovascular function, and immune support.

### Pre-Race Paste Supplement

Generally, a pre-race paste supplement composition may be admistered to a horses a predetermined time (e.g., 30-60 minutes) before a competition. In some cases, the pre-race paste may help move mucous through their respiratory system faster, help coat their stomach to avoid discomfort from stomach acids from nerves, etc. and other anti-inflammatory and calming factors. This may be similar to a pre-workout in that it is an equine preparatory product for top competition. The pre-race paste supplement may be similar to a human daily supplement or all-in-one multi-vitamin-and-mineral essentially to address systemic inflammation.

The pre-race paste may include, in equal parts by weight: a milk thistle component, a licorice root component, and a slippery elm component.

### Top Dressing Supplement

Generally, an oil based top dressing supplement may be used to mix a powder product (e.g., a daily supplement) in with grain feed to horses. The top dressing equine supplemental composition may include an oil component and a honey component.

The honey component may include manuka honey. Manuka honey is known for its anti-inflammatory and antibacterial properties, beneficial to the overall health of horses.

The oil component may include a flax seed oil component and/or a coconut oil component. Flaxseed oil is a rich source of omega-3 fatty acids, important for maintaining a shiny coat and healthy skin in horses. Coconut oil provides medium-chain triglycerides, which are readily used as an energy source by horses.

### EPM Supplement

Generally, an EPM supplement may be administered daily for use to address treating EPM, a neurological disorder that is very common in horses and daily maintenance is required. The EPM supplement may be a high grade combination of Vitamin E and Selenium. Thus, an EPM supplement composition may include a vitamin E component and a selenium component.

In some cases, the EPM supplement composition may further include any one or combinations of: a slippery elm component, a milk thistle component, a spirulina component, a marshmallow root component, a licorice root component, an alfalfa leaf component, an ashwaganda component, a lysine component, a MSM component, and a goji berry component.

### Daily Detoxification Support Product

In some cases, the composition may be a daily detoxification support product designed for daily use. This product comprises specific ingredients in predetermined ratios to support the horse's natural detoxification processes. The components of daily detoxification support product may include:

| Ratio | Component |
|---|---|
| 0.2000 | Vitamin E |
| 0.0667 | Selenium |
| 0.0667 | Goji Berry |
| 0.3333 | MSM |
| 0.3333 | Bentonite Clay |
| 1.0000 | Total |

1. Vitamin E: A fat-soluble antioxidant that helps protect cells from oxidative damage and supports immune function.

2. Selenium: A trace mineral that works synergistically with Vitamin E to enhance antioxidant protection and support overall health.

3. Goji Berry: A nutrient-dense fruit known for its antioxidant properties and potential to support immune function.

4. MSM (Methylsulfonylmethane): An organic sulfur compound that may help reduce inflammation and support joint health.

5. Bentonite Clay: A natural clay with detoxifying properties, which may help bind and remove toxins from the horse's digestive system.

The ratios of these components are carefully balanced to provide optimal support for the horse's daily detoxification needs. This daily detoxification support product may complement the other components of the equine supplemental composition, enhancing the overall health benefits for the horse.

### Hydration Product

In some cases, the composition may be a hydration product. The hydration product may include:

| Ratio | Component |
|---|---|
| 0.333 | Goji Berry |
| 0.333 | Tart Cherry |
| | Powder |
| 0.333 | Salt |
| 1.000 | Total |

The hydration product may be designed to support equine hydration needs. This hydration product is formulated to be versatile in its application, similar to the pre-race paste supplement composition previously described. It can be administered as a powder mixed with water to form a paste, or alternatively, fed directly as a powder on grain or other feed.

The hydration product comprises three key components in equal ratios:

1. Goji Berry: Goji berries, also known as wolfberries, are nutrient-dense fruits rich in antioxidants, vitamins, and minerals. In this hydration product, goji berries may contribute to overall hydration by providing essential electrolytes and supporting cellular health.

2. Tart Cherry Powder: Tart cherries are known for their anti-inflammatory properties and high antioxidant content. In the context of equine hydration, tart cherry powder may help reduce exercise-induced inflammation and support muscle recovery, which can be beneficial for maintaining proper hydration levels during and after physical exertion.

3. Salt: Salt, or sodium chloride, is a crucial component for maintaining proper electrolyte balance in horses. It plays a vital role in fluid retention and helps regulate various bodily functions. The inclusion of salt in this hydration product ensures that horses receive an adequate supply of sodium and chloride ions, which are essential for maintaining proper hydration status.

The equal ratios of these components (0.333 each, totaling 1.000) provides a balanced approach to addressing equine hydration needs. This formulation aims to provide a comprehensive hydration solution that not only replaces lost electrolytes but also offers additional nutritional benefits through the inclusion of goji berries and tart cherry powder.

By incorporating this hydration product into the overall equine supplemental composition, horse owners and trainers can have a versatile tool to support their animals' hydration needs, particularly in situations of increased physical exertion or environmental stress. The powder form allows for easy administration and can be adjusted based on individual horse needs and specific circumstances.

### Terminology

The terminology used above may be interpreted in its broadest reasonable manner, even though it is being used in conjunction with a detailed description of certain specific examples of the present disclosure. Indeed, certain terms may even be emphasized above; however, any terminology intended to be interpreted in any restricted manner will be overtly and specifically defined as such in this Detailed Description section. Both the foregoing general description and the detailed description are exemplary and explanatory only and are not restrictive of the features, as claimed.

As used herein, the terms “comprises,” “comprising,” “having,” including,” or other variations thereof, are intended to cover a non-exclusive inclusion such that a process, method, article, or apparatus that comprises a list of elements does not include only those elements, but may include other elements not expressly listed or inherent to such a process, method, article, or apparatus.

In this disclosure, relative terms, such as, for example, “about,” “substantially,” “generally,” and “approximately” are used to indicate a possible variation of +10% in a stated value.

The term “exemplary” is used in the sense of “example” rather than “ideal.” As used herein, the singular forms “a,” “an,” and “the” include plural reference unless the context dictates otherwise.

### EXAMPLES

Exemplary embodiments of the systems and methods disclosed herein are described in the numbered paragraphs below.

A1. An equine supplemental composition, the composition comprising: a first portion including one or combinations of: a slippery elm component, a milk thistle component, a spirulina component, a marshmallow root component, a licorice root component, an alfalfa leaf or moringa leaf component, an ashwaganda component; and a second portion including one or combinations of: a lysine component, a MSM component, and a goji berry component.

A2. The composition of A1, wherein the first and second portions having ratios, by weight to a total weight of the composition, as follows:

| Ratio | Component |
|---|---|
| 0.07 | Slippery Elm |
| 0.02 | Milk Thistle |
| 0.05 | Spirulina |
| 0.12 | Marshmallow Root |
| 0.05 | Licorice Root |
| 0.07 | Alfalfa Leaf or |
| | moringa leaf |
| 0.07 | Ashwaganda |
| 0.12 | Lysine |
| 0.39 | MSM |
| 0.04 | Goji Berry |

A3. The composition of A1, wherein the second portion includes the lysine component, the MSM component, and the goji berry component.

A4. The composition of A1, wherein the second portion includes the lysine component and the goji berry component.

A5. The composition of A1, wherein the second portion includes the MSM component and the goji berry component.

A6. The composition of A1, wherein the second portion includes the lysine component and the MSM component.

A7. The composition of any of A1-A6, wherein the second portion includes the lysine component with a ratio, by weight to a total weight of the composition, from 0.01 to 0.15.

A8. The composition of any of A1-A6, wherein the second portion includes the MSM component with a ratio, by weight to a total weight of the composition, from 0.20 to 0.60.

A9. The composition of any of A1-A6, wherein the second portion includes the goji berry component with a ratio, by weight to a total weight of the composition, from 0.001 to 0.10.

A10. The composition of any of A1-A9, wherein the first portion includes the slippery elm component, the milk thistle component, the spirulina component, the marshmallow root component, the licorice root component, the alfalfa leaf or moringa leaf component, and the ashwaganda component.

A11. The composition of any of A1-A9, wherein the first portion includes the slippery elm component and one or combinations of: the milk thistle component, the spirulina component, the marshmallow root component, the licorice root component, the alfalfa leaf or moringa leaf component, and the ashwaganda component.

A12. The composition of any of A1-A9, wherein the first portion includes the milk thistle component and one or combinations of: the slippery elm component, the spirulina component, the marshmallow root component, the licorice root component, the alfalfa leaf or moringa leaf component, and the ashwaganda component.

A13. The composition of any of A1-A9, wherein the first portion includes the spirulina component and one or combinations of: the slippery elm component, the milk thistle component, the marshmallow root component, the licorice root component, the alfalfa leaf or moringa leaf component, and the ashwaganda component.

A14. The composition of any of A1-A9, wherein the first portion includes the marshmallow root component and one or combinations of: the slippery elm component, the milk thistle component, the spirulina component, the licorice root component, the alfalfa leaf or moringa leaf component, and the ashwaganda component.

A15. The composition of any of A1-A9, wherein the first portion includes the licorice root component and one or combinations of: the slippery elm component, the milk thistle component, the spirulina component, the marshmallow root component, the alfalfa leaf or moringa leaf component, and the ashwaganda component.

A16. The composition of any of A1-A9, wherein the first portion includes the alfalfa leaf or moringa leaf component and one or combinations of: the slippery elm component, the milk thistle component, the spirulina component, the marshmallow root component, the licorice root component, and the ashwaganda component.

A17. The composition of any of A1-A9, wherein the first portion includes the ashwaganda component and one or combinations of: the slippery elm component, the milk thistle component, the spirulina component, the marshmallow root component, the licorice root component, and the alfalfa leaf or moringa leaf component.

A18. The composition of any of A1-A17, wherein the first portion includes the slippery elm component with a ratio, by weight to a total weight of the composition, from 0.01 to 0.20.

A19. The composition of any of A1-A17, wherein the first portion includes the milk thistle component with a ratio, by weight to a total weight of the composition, from 0.001 to 0.10.

A20. The composition of any of A1-A17, wherein the first portion includes the spirulina component with a ratio, by weight to a total weight of the composition, from 0.01 to 0.10.

A21. The composition of any of A1-A17, wherein the first portion includes the marshmallow root component with a ratio, by weight to a total weight of the composition, from 0.01 to 0.20.

A22. The composition of any of A1-A17, wherein the first portion includes the licorice root component with a ratio, by weight to a total weight of the composition, from 0.01 to 0.10.

A23. The composition of any of A1-A17, wherein the first portion includes the alfalfa leaf or moringa leaf component with a ratio, by weight to a total weight of the composition, from 0.01 to 0.20.

A24. The composition of any of A1-A17, wherein the first portion includes the ashwaganda component with a ratio, by weight to a total weight of the composition, from 0.01 to 0.20.

B1. An equine supplemental composition, the composition comprising: equal parts of each of: a milk thistle component, a licorice root component, and a slippery elm component.

C1. An equine supplemental composition, the composition comprising: an oil component and a honey component.

C2. The composition of C1, wherein the honey component includes manuka honey.

C3. The composition of C1 or C2, wherein the oil component includes a flax seed oil component and a coconut oil component.

D1. An equine supplemental composition, the composition comprising: a vitamin E component, a selenium component, and one or combinations of: a slippery elm component, a milk thistle component, a spirulina component, a marshmallow root component, a licorice root component, an alfalfa leaf or moringa leaf component, an ashwaganda component, a lysine component, a MSM component, and a goji berry component.

D2. The composition of D1, wherein the composition includes the MSM component and the goji berry component.

E1. An equine hydration product comprising: a goji berry component; a tart cherry powder component; and a salt component.

E2. The equine hydration product of E1, wherein the goji berry component, the tart cherry powder component, and the salt component are present in substantially equal ratios.

E3. The equine hydration product of any of E1-E2, wherein each of the goji berry component, the tart cherry powder component, and the salt component has a ratio of about 0.333 by weight to a total weight of the product.

E4. The equine hydration product of any of E1-E3, wherein the product is in a powder form.

E5. The equine hydration product of any of E1-E4, wherein the product is configured to be mixed with water to form a paste.

E6. The equine hydration product of any of E1-E5, wherein the product is configured to be fed directly as a powder on grain or other feed.

F1. An equine detoxification support product comprising: a vitamin E component; a selenium component; a goji berry component; a MSM component; and a bentonite clay component.

F2. The product of F1, wherein the vitamin E component, the selenium component, the goji berry component, the MSM component, and the bentonite clay component are present in predetermined ratios.

F3. The product of any of F1-F2, wherein the vitamin E component has a ratio of about 0.2000 by weight to a total weight of the product.

F4. The product of any one of F1-F3, wherein the selenium component has a ratio of about 0.0667 by weight to a total weight of the product.

F5. The product of any one of F1-F4, wherein the goji berry component has a ratio of about 0.0667 by weight to a total weight of the product.

F6. The product of any of F1-F5, wherein the MSM component has a ratio of about 0.3333 by weight to a total weight of the product.

F7. The product of any of F1-F6, wherein the bentonite clay component has a ratio of about 0.3333 by weight to a total weight of the product.

Other aspects of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An equine supplemental composition, the composition comprising:
a first portion including one or combinations of:
a slippery elm component,
a milk thistle component,
a spirulina component,
a marshmallow root component,
a licorice root component,
an alfalfa leaf or moringa leaf component,
an ashwaganda component; and
a second portion including one or combinations of:
a lysine component,
a MSM component, and
a goji berry component.

2. The composition of claim 1, wherein the first and second portions having ratios as follows:

| Ratio | Component |
|---|---|
| 0.07 | Slippery Elm |
| 0.02 | Milk Thistle |
| 0.05 | Spirulina |
| 0.12 | Marshmallow Root |
| 0.05 | Licorice Root |
| 0.07 | Alfalfa Leaf or |
| | Moringa Leaf |
| 0.07 | Ashwaganda |
| 0.12 | Lysine |
| 0.39 | MSM |
| 0.04 | Goji Berry |

by weight to a total weight of the composition.

3. The composition of claim 1, wherein the second portion includes the lysine component, the MSM component, and the goji berry component.

4. The composition of claim 1, wherein the second portion includes the lysine component and the goji berry component.

5. The composition of claim 1, wherein the second portion includes the MSM component and the goji berry component.

6. The composition of claim 1, wherein the second portion includes the lysine component and the MSM component.

7. The composition of claim 1, wherein the second portion includes the lysine component with a ratio, by weight to a total weight of the composition, from 0.01 to 0.15.

8. The composition of claim 1, wherein the second portion includes the MSM component with a ratio, by weight to a total weight of the composition, from 0.20 to 0.60.

9. The composition of claim 1, wherein the second portion includes the goji berry component with a ratio, by weight to a total weight of the composition, from 0.001 to 0.10.

10. The composition of claim 1, wherein the first portion includes the slippery elm component, the milk thistle component, the spirulina component, the marshmallow root component, the licorice root component, the alfalfa leaf or moringa leaf component, and the ashwaganda component.

11. The composition of claim 1, wherein the first portion includes the slippery elm component and one or combinations of: the milk thistle component, the spirulina component, the marshmallow root component, the licorice root component, the alfalfa leaf or moringa leaf component, and the ashwaganda component.

12. The composition of claim 1, wherein the first portion includes the milk thistle component and one or combinations of: the slippery elm component, the spirulina component, the marshmallow root component, the licorice root component, the alfalfa leaf or moringa leaf component, and the ashwaganda component.

13. The composition of claim 1, wherein the first portion includes the spirulina component and one or combinations of: the slippery elm component, the milk thistle component, the marshmallow root component, the licorice root component, the alfalfa leaf or moringa leaf component, and the ashwaganda component.

14. The composition of claim 1, wherein the first portion includes the marshmallow root component and one or combinations of: the slippery elm component, the milk thistle component, the spirulina component, the licorice root component, the alfalfa leaf or moringa leaf component, and the ashwaganda component.

15. The composition of claim 1, wherein the first portion includes the licorice root component and one or combinations of: the slippery elm component, the milk thistle component, the spirulina component, the marshmallow root component, the alfalfa leaf or moringa leaf component, and the ashwaganda component.

16. The composition of claim 1, wherein the first portion includes the alfalfa leaf or moringa leaf component and one or combinations of: the slippery elm component, the milk thistle component, the spirulina component, the marshmallow root component, the licorice root component, and the ashwaganda component.

17. The composition of claim 1, wherein the first portion includes the ashwaganda component and one or combinations of: the slippery elm component, the milk thistle component, the spirulina component, the marshmallow root component, the licorice root component, and the alfalfa leaf or moringa leaf component.

18. The composition of claim 1, wherein the first portion includes the slippery elm component with a ratio, by weight to a total weight of the composition, from 0.01 to 0.20.

19. The composition of claim 1, wherein the first portion includes the milk thistle component with a ratio, by weight to a total weight of the composition, from 0.001 to 0.10.

20. The composition of claim 1, wherein the first portion includes the spirulina component with a ratio, by weight to a total weight of the composition, from 0.01 to 0.10.
